(19) United States
(12) Patent Application Publication
(10) Pub. No.: US 2025/0312553 A1
(43) Pub. Date: Oct. 9, 2025

(54) Appleman Airway

(71) Applicant: University of Cincinnati, Cincinnati, OH (US)

(72) Inventor: Gregory M. Appleman, Cincinnati, OH (US)

(73) Assignee: University of Cincinnati, Cincinnati, OH (US)

(21) Appl. No.: 18/865,531

(22) PCT Filed: May 15, 2023
(86) PCT No.: PCT/US2023/022240

§ 371 Date: Nov. 13, 2024

Related U.S. Application Data
(60) Provisional application No. 63/341,577, filed on May 13, 2022.

(51) Int. Cl. A61M 16/10; A61M 16/04

(52) U.S. Cl. CPC A61M 16/104; A61M 16/0493

(57) ABSTRACT

An oral airway for use with a sedated patient is provided. The oral airway includes an elongate shaped member with a lip protector that is operatively associated with the proximal end of the elongate shaped member. The oral airway further includes a bite block that is incorporated around at least a portion of the elongate shaped member. The oral airway further includes a first channel and a second channel, both attached to the oral airway. When a scope is inserted through the oral airway, the elongate shaped member is positioned between the tongue and the scope.

### CROSS-REFERENCE TO RELATED APPLICATIONS

This application claims priority to, and the benefit of the filing date of, U.S. Patent Application Ser. No. 63/341,577, filed on May 13, 2022, the disclosure of which is incorporated by reference herein in its entirety.

### TECHNICAL FIELD

The present invention relates to airways for use with sedated patients.

### BACKGROUND OF THE INVENTION

Anesthesia providers frequently administer medicines to provide a level of deep sedation that is required for certain procedures. This is usually called Monitored Anesthesia Care (“MAC”). MAC is different from a General Anesthetic, which produces a deeper level of unconsciousness and usually involves the placement of a breathing tube or other airway device that permits adequate ventilation of the patient. Tens of millions of anesthetics are performed yearly under MAC.

When deep sedation is administered, it is very common for the patient to develop airway obstruction. This is typically a mechanical obstruction caused by the tongue falling back and blocking the airway, which then prevents the patient from adequately ventilating. When this occurs, anesthesia providers resolve the obstruction with various maneuvers such as jaw lifting or head repositioning. Often the obstruction persists, and an airway is placed to permit ventilation. There are various types of oral and nasal airways.

During deep sedation, patients have oxygen administered through a nasal cannula. The patient breathes in through their nose and receives supplemental oxygen throughout their procedure. The nasal cannula typically has 2 channels. One delivers oxygen and the other samples Carbon Dioxide (CO2) that is exhaled by the patient. Exhaled CO2 is a very important way for the provider to ensure that the patient is adequately ventilating.

When an oral airway is placed in a patient's mouth to relieve airway obstruction, this typically leads to patients breathing through their mouth, instead of their nose. When this happens, medical personnel are no longer able to deliver oxygen and sample CO2 through the nose. Providers often try and shove the nasal cannula into the oral airway, or tape it there, but this is typically challenging. Therefore, a need still exists for an improved airway that can displace the tongue while enabling the delivery of oxygen and sampling of CO2 while it is in place.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

In one aspect of the present invention, an oral airway for use with a sedated patient is provided. The oral airway includes an elongate shaped member having a distal end and a proximal end. In various embodiments, the shaped member may be tubular, rectangular, or an open curved shape. The shaped member, when used on a patient, displaces the patient's tongue. The oral airway also includes a lip protector that is operatively associated with the proximal end of the elongate shaped member. The lip protector prevents advancement of the proximal end into the patient's larynx. The oral airway further includes a bite block that is incorporated around at least a portion of the elongate shaped member. The bite block has a central lumen. The oral airway also includes an orifice in the lip protector that is capable of accommodating a scope useful for endoscopy or a transesophageal echocardiogram (TEE). When a scope is inserted, it can pass through the orifice, the bite block and the elongate shaped member and into the patient's esophagus. The oral airway further includes a first channel attached to the oral airway. The first channel passes through a first hole in the lip protector. Also, the first hole is operatively associated with an attachment for universal oxygen tubing. The oral airway also includes a second channel attached to the oral airway. The second channel passes through a second hole in the lip protector. Also, the second hole is operatively associated with an attachment for universal carbon dioxide tubing. When a scope is inserted through the oral airway, the elongate shaped member is located between the tongue and the scope.

In one embodiment, the elongate shaped member is curved. In another embodiment, the first channel is attached to the side of oral airway that is opposite from the direction of the curve. In addition, the second channel is also attached to the side of oral airway that is opposite from the direction of the curve.

In another aspect of the present invention, a method of preventing airway obstruction in a sedated patient while still providing access for a scope to pass into the patient's esophagus is provided. The method involves placing the oral airway described above into the sedated patient's mouth and displacing the patient's tongue. In one embodiment, the patient is sedated using Monitored Anesthesia Care (MAC) or deep sedation anesthesia.

In another aspect of the present invention, a method to prevent airway obstruction in a sedated patient while being able to monitor end tidal carbon dioxide levels in a patient's oropharynx directly through the device is provided. The method involves placing the oral airway described above into the patient's mouth and displacing the patient's tongue. In one embodiment, the method also involves the step of introducing a scope through the oral airway and into the patient's esophagus. In another embodiment, the patient is sedated using MAC or deep sedation anesthesia.

In another aspect of the present invention, a method to prevent airway obstruction in a sedated patient while being able to deliver oxygen directly into a patient's oropharynx is provided. The method involves placing the oral airway described above into the patient's mouth and delivering oxygen directly through the oral airway device. In one embodiment, the method further includes the step of introducing a scope through the oral airway and into the patient's esophagus. In another embodiment, the patient is sedated using MAC or deep sedation anesthesia.

### BRIEF DESCRIPTION OF THE DRAWINGS

The accompanying drawings, which are incorporated in and constitute a part of this specification, illustrate embodiments of the invention and, together with the general description of the invention given above, and the detailed description given below, serve to explain the principles of the invention. Similar reference numerals are used to indicate similar features throughout the various figures of the drawings.

FIG. 1 is an illustration of a side view of an embodiment of the present invention.

FIG. 2 is an illustration of a front view of an embodiment of the present invention.

FIG. 3 is as illustration showing a patient's head with the device of the present invention inserted.

### DEFINITIONS

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

### DETAILED DESCRIPTION OF THE INVENTION

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention, but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

The present invention involves an improved version of the oral airway. The airway serves multiple functions. It pushes the tongue anteriorly to relieve obstruction. In addition, it has two channels that extend from the base of the oral airway in the mouth opening down to the open space that was created when the tongue was displaced. This allows for better delivery of oxygen to the patient and better sampling of their CO2 exhalation. The device may be placed in a patient's mouth to deal with the airway obstruction that occurs during a MAC and allow improved oxygenation and CO2 sampling of patients. In one embodiment, it is compatible with large probe insertion so that it has utility in sedation cases where oral airways are not currently utilized.

During endoscopy and transesophageal echocardiogram (“TEE”) procedures, a large probe is inserted into the mouth and down the esophagus. A bite block is placed prior to placement of the probe to prevent injury to the patient's teeth and to the equipment. Millions of these procedures are performed each year. Deep sedation (or MAC) is frequently provided for these procedures by an anesthesia team. It is very common for the anesthesia provider to encounter airway obstruction, both from the sedation medicines provided as well as from the large probe that takes up a significant portion of the oropharynx. In these situations, the anesthesia provider often relies on a jaw lift maneuver, which elevated the tongue and helps to create additional space to allow ventilation to occur.

In addition to problems with airway obstruction, the anesthesia provider also often has problems with the delivery of oxygen and sampling of exhaled CO2 as described above. This is usually due to the presence of the endoscopy/TEE probe in the mouth leading to more ventilation occurring through the mouth instead of the nose. The present invention addresses this issue by providing an airway device that can be utilized during endoscopy or TEE procedures to displace the tongue. It also provides a larger space to both accommodate the probe and allow for adequate ventilation. The presence of the oxygen delivery and CO2 sampling channels permits improved oxygenation of the patient and better monitoring of their ventilation.

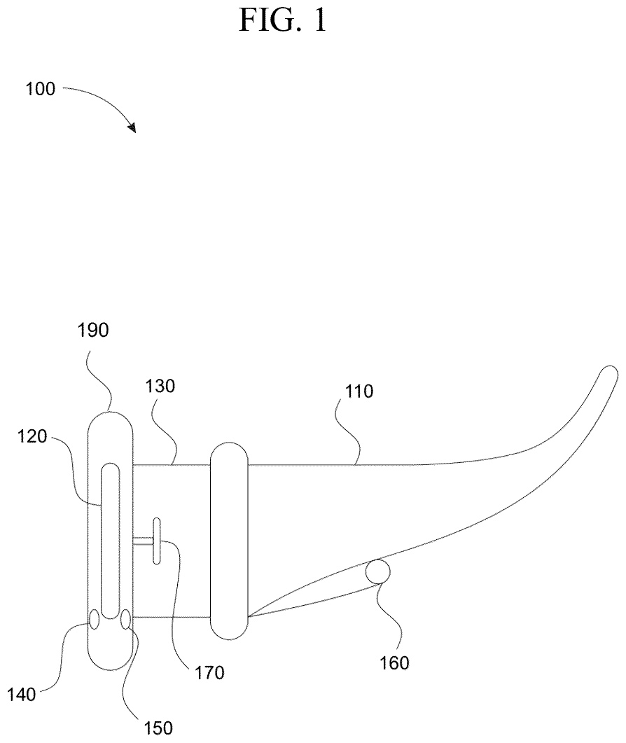

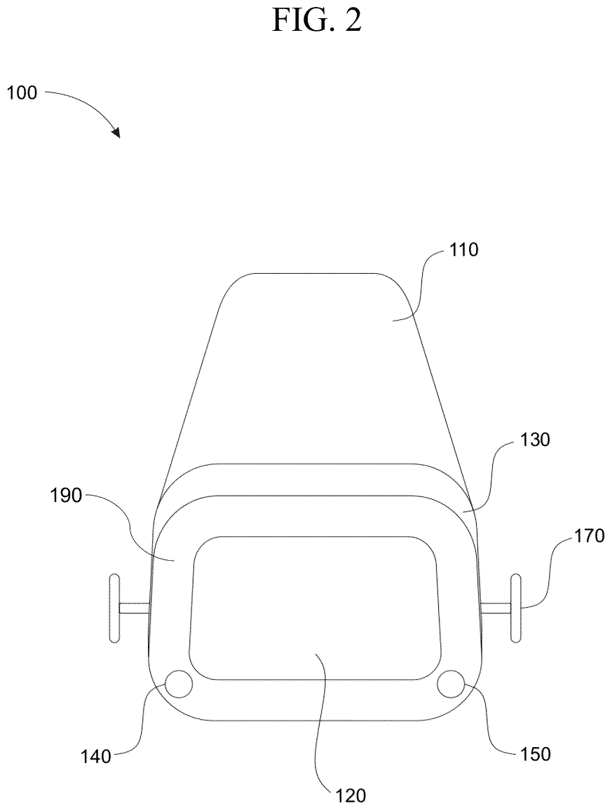

Referring to FIGS. 1 and 2, an embodiment of the device 100 of the present invention is shown. In this embodiment, the device 100 comprises a tongue displacing oral airway 110 with a lip protector 190. The airway 110 is combined with a large bite block 130. The bite block 130 has a central lumen, enabling the passage of one or more scopes through the airway 110. A scope channel 120 is large enough to accommodate probes used for endoscopy and TEE. Probes pass through the airway 110 via the scope channel 120. In addition, this embodiment of the device 100 has two channels, one running along each side of the underside of the oral airway. The channels comprise an oxygen inflow channel 140 and a CO2 sampling channel 150. 160 is the termination of the CO2 sampling channel 150. In one embodiment, the channels are tubing. In one embodiment, these channels terminate about ⅓ of the length of the airway starting at the proximal end of the airway. In one embodiment, the oxygen inflow channel 140 has an attachment at the base to attach to universal oxygen tubing. Also, the CO2 sampling channel 150 has an attachment for universal CO2 sampling tubing. Further, the device 100 has a strap attachment 170 on either side of the device 100. A strap (not shown) is connected to each strap attachment 170. The strap is then placed behind the patient's head to secure the device 100. In one embodiment, the strap is a rubber strap.

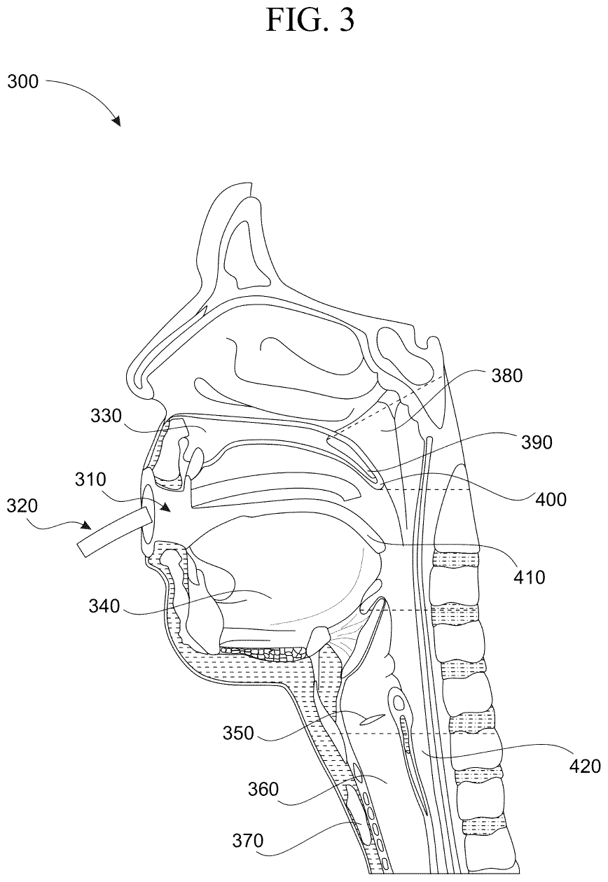

Referring to FIG. 3, an illustration of a patient's head with the airway 300 of the present invention inserted is shown. An embodiment of the airway 310 of the present invention is inserted in the patient's mouth. For reference, the illustration shows the hard palate 330, the tongue 340, the vocal cords 350, the trachea 360, the thyroid 370, the nasopharynx 380, the soft palate 390, the uvula 400 and the esophagus 420. An endoscope 320 is shown passing through the airway 310. The inserted length of the endoscope 320 is located on top of the airway 310. The airway 310 displaces the tongue 340, resulting in the airway 310 being located between the tongue 340 and the endoscope 320 when the endoscope 320 is inserted. The airway 310 is shown displacing the tongue 410.

### Materials

The present invention, in alternative embodiments, may be non-sterile, or sterile, and/or for single use only. In alternative embodiments the device is made of a material that is sterilizable and/or autoclavable for reuse. The device of the present invention, in alternative embodiments, may be reusable or disposable.

In alternative embodiments, the device of the present invention or any components thereof, can be manufactured by any known method, e.g., by injection molding, e.g., of a biologically inert, medical grade, latex free polymer or polymers. In alternative embodiments, devices of the present invention, or any components thereof, are made using biodegradable polymers, polycarbonates, soft or hard polycarbonates, or equivalents.

In alternative embodiments, the device of the present invention or any components thereof, are scaled to different proportional dimensions to accommodate the differing airway sizes of patients of all ages and physical dimensions. Devices according to the present invention can be designed to have optimum dimensional relationships, to include variations in size, materials, shape, form, function and manner of operation, assembly and use, as are deemed readily apparent to one skilled in the art, and all equivalent relationships to those illustrated in the drawings and described in the specification are intended to be encompassed by the present inventive subject matter. In one embodiment, the device of the present invention is included in a kit containing several sizes of the oral airway to fit sizes from a child to a large adult.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. An oral airway for use with a sedated patient, the patient having a tongue, a larynx and an esophagus, the oral airway comprising:
a. an elongate shaped member having a distal end and a proximal end, wherein the shaped member, when used on a patient, displaces the patient's tongue;
b. a lip protector operatively associated with the proximal end of the elongate shaped member, wherein the lip protector prevents advancement of the proximal end into the patient's larynx;
c. a bite block incorporated around at least a portion of the elongate shaped member, the bite block having a central lumen;
d. an orifice in the lip protector that is capable of accommodating a scope useful for endoscopy or a transesophageal echocardiogram (TEE), wherein when a scope is inserted, it can pass through the orifice, the bite block and the elongate shaped member and into the patient's esophagus;
e. a first channel attached to the oral airway, wherein the first channel passes through a first hole in the lip protector and further, wherein the first hole is operatively associated with an attachment for universal oxygen tubing; and
f. a second channel attached to the oral airway, wherein the second channel passes through a second hole in the lip protector and further, wherein the second hole is operatively associated with an attachment for universal carbon dioxide tubing;
wherein, when a scope is inserted through the oral airway, the elongate shaped member is located between the tongue and the scope.

2. The oral airway of claim 1 wherein the elongate shaped member is curved.

3. The oral airway of claim 2 wherein the first channel is attached to the side of oral airway that is opposite from the direction of the curve, and further, wherein the second channel is also attached to the side of oral airway that is opposite from the direction of the curve.

4. A method of preventing airway obstruction in a sedated patient while still providing access for a scope to pass into the patient's esophagus comprising placing the oral airway of claim 1 into the sedated patient's mouth and displacing the patient's tongue.

5. The method of claim 4 wherein the patient is sedated using Monitored Anesthesia Care (MAC) or deep sedation anesthesia.

6. A method to prevent airway obstruction in a sedated patient while being able to monitor end tidal carbon dioxide levels in a patient's oropharynx directly through the device, the method comprising placing the oral airway of claim 1 into the patient's mouth and displacing the patient's tongue.

7. The method of claim 6 further comprising the step of introducing a scope through the oral airway and into the patient's esophagus.

8. The method of claim 6 wherein the patient is sedated using MAC or deep sedation anesthesia.

9. A method to prevent airway obstruction in a sedated patient while being able to deliver oxygen directly into a patient's oropharynx, the method comprising placing the oral airway of claim 1 into the patient's mouth and delivering oxygen directly through the oral airway device.

10. The method of claim 9 further comprising the step of introducing a scope through the oral airway and into the patient's esophagus.

11. The method of claim 9 wherein the patient is sedated using MAC or deep sedation anesthesia.
